(19) United States
(12) Patent Application Publication
(10) Pub. No.: US 2021/0162144 A1
(43) Pub. Date: Jun. 3, 2021

(54) Vaginal Cleansing Device

(71) Applicant: Chung-Sing Lin, Tainan City (TW)

(72) Inventor: Chung-Sing Lin, Tainan City (TW)

(21) Appl. No.: 17/081,028

(22) Filed: Oct. 27, 2020

(30) Foreign Application Priority Data
Nov. 28, 2019 (TW) 108143505

(51) Int. Cl. A61M 13/00

(52) U.S. Cl. CPC A61M 13/003; A61M 2202/02; A61M 2210/1475

(57) ABSTRACT

A vaginal cleansing device is composed of an outer film, an inner support cylinder and a plug. The outer film has conical tapered rings formed on an outer wall of the outer film and tapered upward. The inner support cylinder has microvias formed on a wall of the inner support cylinder and corresponsive to the conical tapered rings of the outer film respectively; the inner support cylinder is plugged into the outer film, so that the microvias of the inner support cylinder are corresponsive to the conical tapered rings of the same height; and the microvias are attached onto the interior of the conical tapered rings. The plug is plugged into the opening of the inner support cylinder tightly and has a gas passing tube passing through the center of the opening of the inner support cylinder and a connector connected to the bottom end of the gas passing tube.

### BACKGROUND OF THE INVENTION

### Field of the Invention

The present invention relates to a vaginal cleansing device, and more particularly to the vaginal cleansing device for cleaning a woman's vaginal wall during or at the end of menstruation.

### Description of the Related Art

The most important mission of a woman's life is to give birth. To prepare for fertility, women are arranged to have periodic ovulation periods and menstrual periods, and normal women starting from the age of 10˜12 and ending at the age of 50 have a menstrual period occurring almost at the same time every month, and the menstrual period is commonly known as menstruation or period. In simple terms, the emergence of a female menstrual period is to prepare for childbirth, and women must welcome sperm acceptance and egg implantation, in which the endometrium will be proliferated and thickened during ovulation, and an egg will be released from the ovary and entered into the uterus to wait for fertilization, and this period is called the ovulation period. However, if the egg has not been fertilized during the ovulation period, the proliferative endometrium will be peeled off and discharged from the vagina, thus forming the menstrual period.

In modern highly-developed societies, everyone has scheduled work every day. Students have class to attend, office workers have office work to do, and even housewives have daily household work to take care every day, so that when women have menstrual periods, they have to bother to keep clean and hygienic. The most commonly used sanitary products during the menstrual period include sanitary napkin and tampon, wherein the sanitary napkin is used to absorb menstrual blood, so that when the amount of menstrual blood is large, the sanitary napkin must be replaced frequently. In addition to the purpose of maintaining cleanliness and hygiene, the most important purpose is to prevent menstrual blood from permeating or leaking to the outside that causes a bad body image. The tampon is used for absorbing menstrual blood as well as providing a blocking effect to prevent menstrual blood from passing through (when needed), so that women will give high priority to deal with the discharge of menstrual blood by using tampons when they participate in certain jobs or occasions.

Using sanitary products during menstruation is actually a passive behavior. In details, the proliferative endometrium is peeled off and discharged from the vagina is an active physiological change of women. In contrast, using the sanitary napkin or tampon to absorb menstrual blood is of course a passive waiting behavior. In fact, women do not discharge menstrual blood continuously all the time during the menstrual period (usually around 7˜10 days). The real situation is a burst of discharge from time to time, and sometimes the amount of discharged menstrual blood will be more, and sometime the amount of discharged menstrual blood will be less, and there are different discharge patterns, and the pattern of menstrual blood discharge of some women varies every time, and thus the menstrual period is quite troublesome for women.

Although the use of sanitary products to wait for (or absorb) menstrual blood during menstruation is a well-known behavior, it is not necessary to use such sanitary products continuously during menstruation. In fact, these sanitary products are mainly used for preventing menstrual blood from leaking when going out that causes a bad body image. Assumed that a woman stays at home or in an environment that she can clean the menstrual blood at any time, the woman can actually choose not to use the sanitary product for a long time or can use it only if there is a lot of menstrual blood or during sleep, or choose not to use it at all when the amount of menstrual blood has been decreased significantly or when no menstrual blood is discharged anymore. The woman can choose not to use the sanitary product at home or in a cleanable environment, so that the vagina and vaginal opening will not be blocked or compressed for a long time to avoid inflammation and maintain cleanliness. In the mind of many women and health professionals, the concept of “unnecessary to use sanitary products continuously during menstruation” has been gradually accepted and recognized by the female public.

In other words, the using amount of sanitary products during menstruation is reduced, not just giving the benefits of saving expenses and reducing wastes only, but also achieving a good effect on body and mind. However, the realization of reducing the use of sanitary products is actually accompanied with the requirement of maintaining cleanliness. During or after a menstrual period, menstrual blood and blood clot will be passed through and adhered to the vagina, and thus it is necessary to clean the vagina in order to keep it clean and hygienic after using the sanitary product or stopping the use of the sanitary product.

### SUMMARY OF THE INVENTION

In view of the necessity of cleansing the vagina during and after menstrual period and menstrual period, the inventor of the present invention conceived and developed a vaginal cleansing device exclusively for women, which can be used for cleaning the vagina in a timely manner. Based on this demand, the inventor conducted extensive research and experiment, and finally developed the vaginal cleansing device in accordance with the present invention.

Therefore, it is a primary objective of the present invention to provide a vaginal cleansing device exclusively for women to clean the vagina timely.

To achieve the above-mentioned objective, the present invention provides a vaginal cleansing device, comprising an outer film, an inner support cylinder and a plug, characterized in that the outer film is made of a flexible material with excellent flexibility and ductility and formed into a long stripe bag shape, and the outer film has a top edge formed into an arc seal and a bottom formed into an opening, and the outer film has a plurality of conical tapered rings formed on an outer wall thereof and made of a same material, and the conical tapered rings are tapered upward, and the outer film has a pull ring formed at the exterior of the opening and situated at a position at least partially higher than the opening, and a part of the pull ring is disposed deeply within a range of the outer film, so that the outer film at the position near the pull ring is inwardly formed into a neck; the inner support cylinder is made of a flexible material and formed into a long cylinder, and the inner support cylinder has a supportive property and the inner support cylinder has a top edge formed into an arc seal and a bottom formed into an opening, and the inner support cylinder has a plurality of microvias formed at position thereof and configured to be corresponsive to the plurality of conical tapered rings of the outer film respectively, and a neck concavely formed at a position close to the opening, and the neck of the inner support cylinder is corresponsive to the neck of the outer film; the plug is made of a flexible material and formed into a circular body with a specific height, and the plug has the periphery concavely formed into a neck, and the neck of the plug is corresponsive to the neck of the inner support cylinder, and the plug has a gas passing tube passing through the center of the plug, and a connector coupled to the bottom end of the gas passing tube, and the plug is plugged into the opening of the inner support cylinder during assembling, and the neck of the plug and the neck of the inner support cylinder are combined tightly with each other, and the top edge of the inner support cylinder is aligned and inserted into the opening of the outer film and entered into the outer film, so that the plurality of microvias with different heights disposed on the inner support cylinder is corresponsive to the conical tapered rings of the same height, and the plurality of microvias is attached to the interior of the corresponding conical tapered rings respectively.

According to the above-mentioned structure and composition, the main purpose of the present invention is to help women clean the vaginal wall and keep the vaginal wall clean, and the timing of use of the vaginal cleansing device is determined according to a user's own judgment, and the device can be used during the menstrual period, after the menstrual period, or at other time points, such as the time after sexual intercourse, which is helpful to contraception and sometimes even when rape occurs, since this vaginal cleansing device can be used to eliminate other substances in the vagina to help prevent unwanted pregnancy.

### BRIEF DESCRIPTION OF THE DRAWINGS

FIG. 1 is a perspective view of a structure of the present invention after assembling;

FIG. 2 is an exploded view showing a half-cut structure of the present invention before assembling;

FIG. 3 is an exploded view of a structure of the present invention before assembling;

FIG. 4 is a longitudinal section view of a structure of the present invention after assembling;

FIGS. 5 and 6 are schematic views showing a continuous process of assembling and using the present invention;

FIG. 7 is a schematic view showing the action of applying the present invention into a women's vagina.

### DETAILED DESCRIPTION OF THE PREFERRED EMBODIMENTS

The present invention will become clearer in light of the following detailed description of an illustrative embodiment of this invention described in connection with the drawings.

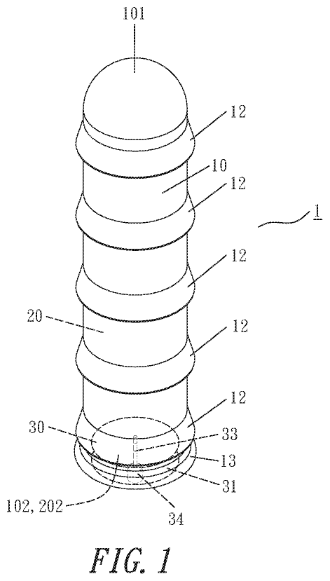

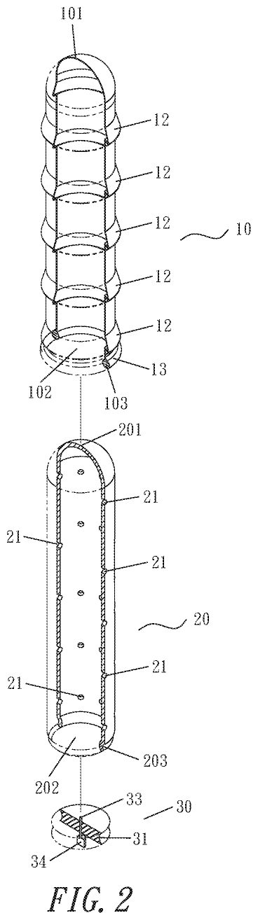

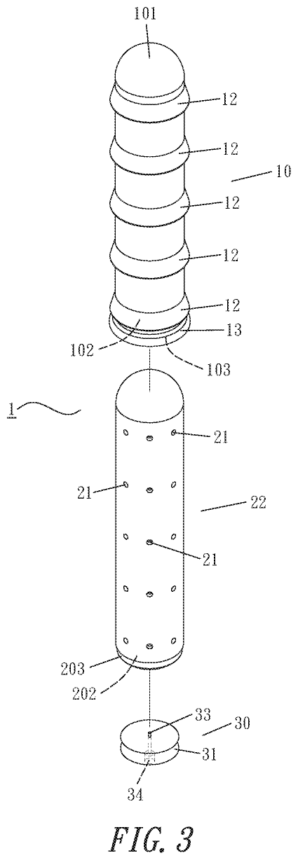

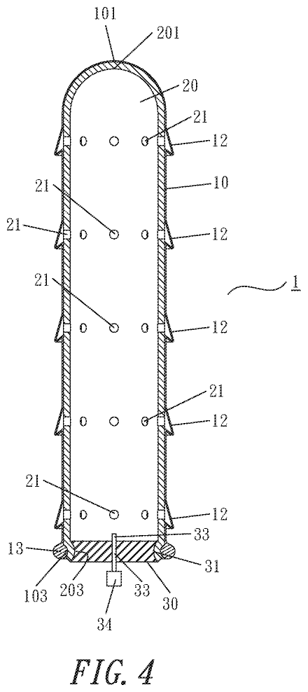

The title of the present invention is “Vaginal cleansing device”, and thus the invention is directed to a vaginal cleansing apparatus exclusively for women. With reference to FIGS. 1˜4 for a vaginal cleansing device 1 of the present invention, the basic structure of the vaginal cleansing device 1 is comprised of an outer film 10, an inner support cylinder 20, and a plug 30, wherein the outer film 10 is made of a flexible material with excellent flexibility and ductility such as latex, silicone, and other composite materials and formed into a long stripe bag shape, and the outer film 10 has a top edge 101 formed into an arc seal and a bottom formed into an opening 102, and the outer film 10 has a plurality of conical tapered rings 12 formed on an outer wall of the outer film 10 and made of a same material, and the conical tapered ring 12 is tapered upward (with respect to the figures), and the outer film 10 has a pull ring 13 formed at the exterior of the opening 102 and situated at position at least partially higher than the opening 102, and a part of the pull ring is disposed deeply within a range of the outer film 10, so that a neck 103 is inwardly formed on the outer film 10 at a position near the pull ring 12. The inner support cylinder 20 is also made of a flexible material with excellent flexibility and ductility such as latex, silicone, and other composite materials and formed into a long cylinder, but the inner support cylinder 20 does not have excellent flexibility and ductility. Although the inner support cylinder 20 is not a rigid body, it must have a relatively high supportive property, and the inner support cylinder 20 has a top edge 201 formed into an arc seal and a bottom formed into an opening 202, and the inner support cylinder 20 has a plurality of conical tapered rings 12 formed on a wall of the inner support cylinder 20 and a plurality of microvias 21 configured to be corresponsive to the outer film 10, so that the microvias 21 are situated at a same height, and the microvias 21 situated at different heights are configured to be corresponsive to a conical tapered ring 12, and the inner support cylinder 20 has a neck 203 concavely formed at a position near the opening 202 and corresponsive to the aforementioned neck 103. The plug 30 is made of a flexible material such as rubber, silicone, and other composite materials and formed into a circular body with a specific height, and the periphery of the plug 30 is concavely formed into a neck 31, and corresponsive to the aforementioned neck 203, and a gas passing tube 33 is passed through the center of the plug 30, and a connector 34 is coupled to the bottom end of the gas passing tube 33.

Before the outer film 10 and the inner support cylinder 20 are assembled (or combined), or the two exist independently, and the outer film 10 is substantially in the shape of a flexible long-stripe bag, and the inner support cylinder 20 is a hard supporting member, and the volume in the outer film 10 is smaller than (or not greater than) the volume enclosed by the wall of the inner support cylinder 20. The volume of the plug 30 can be reduced by compression, so that the plug 30 can be plugged tightly into the opening 202 of the inner support cylinder 20.

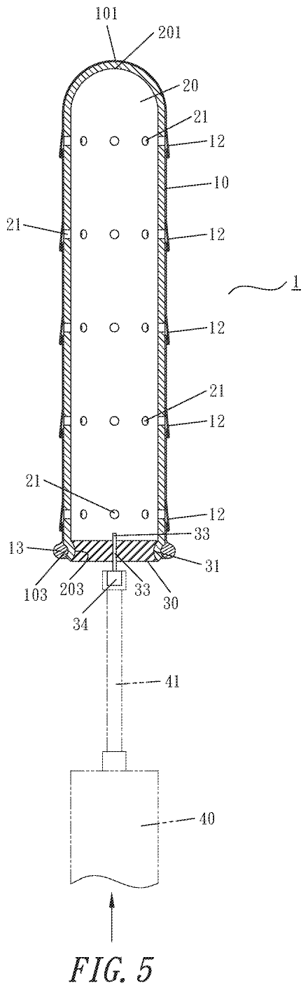

When the vaginal cleansing device 1 is used, the following procedure must be performed to combine the outer film 10, the inner support cylinder 20 and the plug 30 as shown in FIGS. 5˜6 (wherein the inflator 40 and the air outlet tube 41 in the figures are not referred yet). The plug 30 is plugged into the opening 202 of the inner support cylinder 20 by means of squeezing and compressing actions. After the plug 30 is plugged, a tight combination is achieved between an outer wall of the neck 31 of the plug 30 and an inner wall of the neck 203 of the inner support cylinder 20, and then the top edge 201 of the inner support cylinder 20 is held and then aligned with the opening 102 of the outer film 10 before it is inserted into the outer film 10, and the insertion process of the inner support cylinder 20 will push the wall of the outer film 10 to stretch out, so as to allow the inner support cylinder 20 to enter into the outer film 10 step by step, and finally when the outer wall of the top edge 201 of the inner support cylinder 20 reaches the inner wall of the top edge 101 of the outer film 10, the neck 203 of the inner support cylinder 20 will abut against the neck 103 of the outer film 10 correspondingly, while the microvias 21 of different heights disposed on the inner support cylinder 20 are aligned with the conical tapered rings 12 of the same height, and the microvias 21 will be attached to the interior of the conical tapered rings 12 respectively, so that all microvias 21 of the inner support cylinder 20 are now covered by the outer film 10 and cannot be communicated to the outside, and only the gas passing tube 33 and the connector 34 of the inner support cylinder 20 can be communicated to the outside. Now, the outer film 10, the inner support cylinder 20 and the plug 30 are combined to form a vaginal cleansing device 1, and the perspective view of the vaginal cleansing device 1 is shown in FIG. 1.

Since the vaginal cleansing device 1 must be used in a clean and hygienic condition, therefore isolated packaging should be done immediately after the manufacturing and assembling processes are completed. For example, an outer surface of the assembled vaginal cleansing device 1 is coated with a shrinkable film that serves as an inner package (which is an inner protective layer), and then a rubber cover is placed into the coated vaginal cleansing device 1 to serve as an outer package (which is an outer protective layer). More specifically, before the isolated packaging of the vaginal cleansing device 1, a little amount of lubricating gel is coated onto an outer surface of the outer film 10 (including the conical tapered ring 12), so that when the isolated packaging is opened to take out the vaginal cleansing device 1, we can feel that there is a little lubricating gel on the outer membrane body 10 (including the conical tapered ring 12) of the vaginal cleansing device 1.

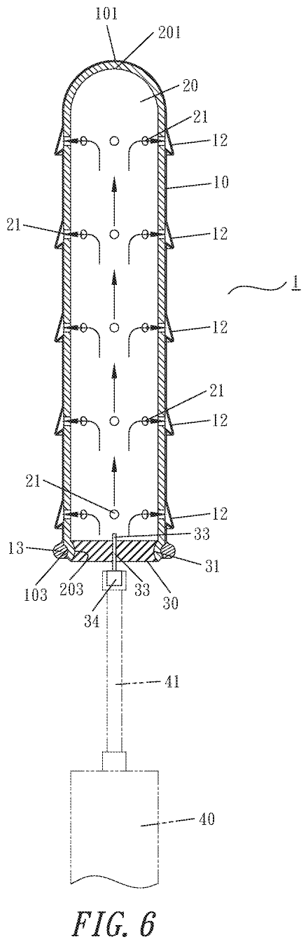

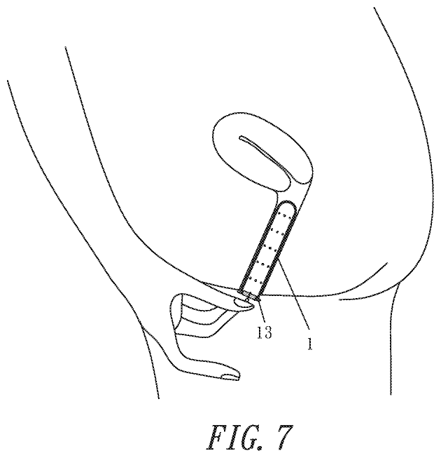

During use as shown in FIG. 7, a vaginal cleansing device 1 is held, and aligned precisely and inserted from the top edge 101 of the outer film 10 into the vaginal opening, so that the whole vaginal cleansing device 1 is penetrated and remained deeply into an appropriate position (near the cervix), and the lubricating gel on the outer surface of the vaginal cleansing device 1 or the lubricating gel applied by users allows the insertion process to be completed smoothly and successfully. After the vaginal cleansing device 1 reaches a fixed position, a common part of the neck 103 of the pull ring 13 and the neck 203 must stay and wait outside the vaginal opening, and then an end of an air outlet tube 41 of an inflator 40 is coupled to an external connector 34 of the plug 30. As shown in FIGS. 5 and 6, the inflator 40 is not an element claimed by the present invention, and any inflator that can be connected to the connector 34 can be used. The inflator 40 is operated to pump gas into the inner support cylinder 20 through the connector 34 and the gas passing tube 33 to increase the pressure. In this process, the gas inside the inner support cylinder 20 is ejected to the outside through the plurality of microvias 21 to push and squeeze the plurality of conical tapered rings 12 to change from its original dispirited state (as shown in FIG. 5) into an erect state (as shown in FIG. 6). In the meantime, the outer film 10 is already in an expanded state; so that when a user feels (based on accumulated experience) that the air pressure inside the inner support cylinder 20 has reached an appropriate level, the user stops the air inflation of the inflator 40 and remove the inflator 40 together with the air outlet tube 41. Now, the pressure inside the inner support cylinder 20 and the outer film 10 can be maintained by the unidirectional effect of the connector 34. After the inflator 40 together with the air outlet tube 41 is removed, the vaginal cleansing device 1 in the expanded state stays in the vagina. Finally, the user holds the pull ring 13 by the palm to pull the whole vaginal cleansing device 1 slowly outward and away from the vagina. In this process, the plurality of conical tapered rings 12 in the erect state will slide across the vaginal wall to carry out the menstrual blood and blood clot attached on the vaginal wall, so as to clean the vagina.

After the use of the vaginal cleansing device 1 is completed, the connector 34 is pressed to release the gas inside the inner support cylinder 20 and the outer film 10, and then the user can choose to remove the outer film 10 from the inner support cylinder 20 and throw away the outer film 10. A new outer film 10 is installed for the next time of use, or the used outer film 10 is washed and cleaned directly, and then the whole vaginal cleansing device 1 is cleaned and stored for the next time of use. The vaginal cleansing device 1 can be reused after an appropriate hygiene treatment (such as disinfection by alcohol), which is just a choice of use.

From the description above, the present invention provides a novel vaginal cleansing device 1 comprised of an outer film 10, an inner support cylinder 20 and a plug 30 and having a main purpose to help women clean the vaginal wall and keep the vaginal wall clean, so that the timing of use is determined by the user's own judgment. For example, the vaginal cleansing device 1 can be used during menstrual period, after menstrual period, after sexual intercourse, or even after the woman has been raped. The vaginal cleansing device 1 can eliminate other substances in the vagina and help prevent unwanted pregnancy.

In summation of the description above, the present invention can achieve the expected functions and objectives, and the detailed description of the invention allows people having ordinary skill in the art to implement the invention accordingly. While the invention has been described by way of examples and in terms of preferred embodiments, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. A vaginal cleansing device, comprising an outer film, an inner support cylinder and a plug, characterized in that the outer film is made of a flexible material with excellent flexibility and ductility and formed into a long stripe bag shape, and the outer film has a top edge formed into an arc seal and a bottom formed into an opening, and the outer film has a plurality of conical tapered rings formed on an outer wall thereof and made of a same material, and the conical tapered rings are tapered upward, and the outer film has a pull ring formed at the exterior of the opening and situated at a position at least partially higher than the opening, and a part of the pull ring is disposed deeply within a range of the outer film, so that the outer film at the position near the pull ring is inwardly formed into a neck; the inner support cylinder is made of a flexible material and formed into a long cylinder, and the inner support cylinder has a supportive property and the inner support cylinder has a top edge formed into an arc seal and a bottom formed into an opening, and the inner support cylinder has a plurality of microvias formed at position thereof and configured to be corresponsive to the plurality of conical tapered rings of the outer film respectively, and a neck concavely formed at a position close to the opening, and the neck of the inner support cylinder is corresponsive to the neck of the outer film; the plug is made of a flexible material and formed into a circular body with a specific height, and the plug has the periphery concavely formed into a neck, and the neck of the plug is corresponsive to the neck of the inner support cylinder, and the plug has a gas passing tube passing through the center of the plug, and a connector coupled to the bottom end of the gas passing tube, and the plug is plugged into the opening of the inner support cylinder during assembling, and the neck of the plug and the neck of the inner support cylinder are combined tightly with each other, and the top edge of the inner support cylinder is aligned and inserted into the opening of the outer film and entered into the outer film, so that the plurality of microvias with different heights disposed on the inner support cylinder is corresponsive to the conical tapered rings of the same height, and the plurality of microvias is attached to the interior of the corresponding conical tapered rings respectively.
